(19) United States
(12) Patent Application Publication
(10) Pub. No.: US 2012/0264628 A1
(43) Pub. Date: Oct. 18, 2012

(54) Methods for Enriching Microparticles or Nucleic Acids in a Complex Mixture Using Size Exclusion Filtration

(75) Inventors: Patricia Okamoto, Shrewsbury, MA (US); Jan Godoski, Seattle, WA (US); Thomas Scholl, Westborough, MA (US)

(73) Assignee: Esoterix Genetic Laboratories, LLC, Burlington, NC (US)

(21) Appl. No.: 13/362,655

(22) Filed: Jan. 31, 2012

Related U.S. Application Data
(60) Provisional application No. 61/437,847, filed on Jan. 31, 2011.

(51) Int. Cl. C07H 1/08; G01N 21/64; C07H 21/04; C40B 30/04; G01N 27/62; G01N 27/26; C12Q 1/68; G01N 21/76

(52) U.S. Cl. 506/9; 435/6.12; 435/6.11; 435/6.14; 536/25.4

(57) ABSTRACT

Embodiments of the present invention provide methods for the enrichment of rare microparticles, cells, or nucleic acids from a complex mixture using serial size exclusion filtration. Also provided are less invasive methods for detecting chromosomal or genetic abnormalities in a fetus, by enriching fetal microparticles in maternal plasma using serial size exclusion filtration, and isolating and analyzing the fetal nucleic acids from the fetal microparticles. Methods for diagnosis of diseases such as cancer are also provided, including enriching disease specific microparticles in the patient's plasma using serial size exclusion filtration, and isolating and analyzing the nucleic acids from the disease specific microparticles.

### PRIOR RELATED APPLICATION

This application claims priority to U.S. provisional application No. 61/437,847, filed Jan. 31, 2011, the contents of which are hereby incorporated by reference in their entirety.

### FIELD

Embodiments of the present invention relate to methods for enriching a rare population of microparticles, cells, or nucleic acids from a complex mixture, such as blood.

### BACKGROUND

Assessing and monitoring fetal health are of utmost importance during a pregnancy. Doctors and other medical professionals need to have the most accurate information available regarding the health of the fetus in order to minimize the risks to both the fetus and the mother during pregnancy and to optimize the number of healthy babies born. Understandably, expectant parents and relatives are also anxious for information about the health and condition of the fetus. It is desirable for this information to be available as early as possible so that the parents may make informed decisions regarding the pregnancy and any adverse medical conditions the fetus may have.

Access to fetal genetic material can provide significant information regarding the health of the fetus. For example, any genetic defects, such as chromosomal abnormalities, can be detected by analyzing fetal DNA. Chromosomal abnormalities include point substitutions, deletions, additions, translocations, or abnormal numbers of chromosomes or chromosome sets (aneuploidy). One example of aneuploidy is monosomy, a type of aneuploidy in which one chromosome of a pair is missing. Another type of aneuploidy is trisomy, in which there are three copies of a chromosome instead of a pair. Aneuploidy may be lethal or may cause one of several different genetic disorders, including Down syndrome (Trisomy 21), Edwards syndrome (Trisomy 18), Patau syndrome (Trisomy 13), and Turner syndrome (X instead of XX or XY).

For prenatal diagnosis of these conditions, the currently available procedures are limited and have certain disadvantages. One currently used procedure is amniocentesis, a medical procedure in which amniotic fluid containing fetal DNA is extracted from the amniotic sac where the fetus is developing, and then the fetal DNA is analyzed for any genetic abnormalities. Amniocentesis is usually performed between the fifteenth and twentieth week of the pregnancy (i.e., during the second trimester). Amniocentesis carries the risk of several significant complications, including preterm labor, fetal trauma, and even miscarriage of the fetus. Because the test cannot be performed reliably until the second trimester of the pregnancy, and because of the significant risks associated with the procedure, amniocentesis may not be a desirable procedure for many patients. Another procedure that is currently used is chorionic villus sampling (CVS), in which a sample of the placental tissue is taken and analyzed. CVS can be performed earlier than an amniocentesis (i.e., typically between 10-12 weeks of the pregnancy), but this procedure also carries increased risk of infection, fetal trauma, amniotic fluid leakage, and miscarriage. CVS is also subject to maternal cell contamination if maternal cells are not completely separated from the placenta. Therefore, because both amniocentesis and CVS are relatively invasive procedures and have certain health risks and disadvantages, these procedures may not be suitable for many patients.

Some fetal material is also present in the mother's bloodstream. This material includes fetal DNA contained in microparticles (also called vesicles, microvesicles, or apoptotic bodies) that are formed primarily when placental cells undergo apoptosis or other forms of cell death. Morphological changes occur during apoptosis or other forms of cell death, including a process known as “membrane blebbing,” which leads to the formation and release of these microparticles from the cell. Because these microparticles are formed from the cell membrane, the microparticles have on their surface biomarkers that are specific for the cell from which they formed. In addition, the contents of the microparticle can include nuclear material such as nucleic acids that are specific for the cell from which they were released. The sizes of the microparticles and the amount of microparticles present in the mother's bloodstream may vary based on the individual and, to a lesser extent, based on the gestational age of the fetus. In some instances, the amount of microparticles present may be correlated with adverse conditions during the pregnancy. Generally, the average size of the microparticles ranges from about 0.1 to about 1 μm. These microparticles are only present in the maternal bloodstream in very small amounts, and it is extremely difficult using known methods to distinguish the fetal DNA from the maternal DNA. If the fetal DNA could be isolated or purified, however, valuable information regarding the health of the fetus, including information about chromosomal or genetic abnormalities, could be obtained without imposing significant health risks to the mother or the fetus.

The isolation and enrichment of microparticles have other applications as well. For example, microparticles are formed during the activation or apoptosis or other types of cell death of cancer cells, or the activation or apoptosis or other cell death of cells in certain other diseases. In addition, in patients that have cancer (and likely other diseases), microparticles are released from the cells not only during cell death, but also intentionally by the cells, for example, during metastasis of the cancer. These disease specific microparticles may be found circulating in the patient's bloodstream or in other bodily fluids that come into contact with the disease or cancer cells.

Therefore, what is needed is a less invasive and reliable method for detecting fetal chromosomal or other genetic abnormalities of a fetus early in a pregnancy (i.e., during the first trimester). It is also desirable for such a method to be accurate, sensitive, and reproducible throughout the pregnancy (e.g., for monitoring the health of the fetus throughout pregnancy). Methods for enriching fetal microparticles and fetal DNA from maternal material are also needed. These methods are preferably efficient, informative, and inexpensive. What is also needed is a method to enrich disease specific microparticles (e.g., cancer microparticles) or the nucleic acids contained in such microparticles in order to detect, monitor, and analyze diseases, tumors, or other cancers.

### SUMMARY

Certain embodiments of the present invention provide methods of size exclusion via filtration to enrich for microparticles that contain nucleic acids from a complex mixture such as blood. Microparticles (also known as microvesicles, vesicles, or apoptotic bodies) containing nucleic acids have been reported to range in size from about 0.1 μm to about 1 μm. Based on their wide range of sizes, these microparticles can be selectively captured on filters of various diameter pores. Once captured, the microparticles may be solubilized, and the nucleic acids can be isolated directly from the membrane using standard molecular biological methods.

This method has particular application in the enrichment of fetal microparticles or fetal DNA from the plasma of pregnant women. Previous studies have reported that fetal DNA-associated microparticles are released into maternal plasma during pregnancy and are present at very low amounts in the maternal plasma. Thus, enrichment for these microparticles would allow for the enrichment and isolation of pure fetal DNA from maternal blood. The capture of microparticles by size filtration provides a simple, fast, and cost-effective method for the enrichment of fetal DNA from maternal plasma.

In certain aspects, methods for enriching fetal microparticles in a biological sample are provided, including the steps of passing a biological sample through a first membrane having a first membrane pore size, wherein the sample is separated into a first flowthrough fraction and a first membrane fraction; passing the first flowthrough fraction through a second membrane with a pore size that is smaller than the first membrane pore size, wherein the first flowthrough fraction is separated into a second flowthrough fraction and a second membrane fraction; and wherein the fetal microparticles are enriched in at least one of the four flowthrough and membrane fractions. In certain embodiments, the biological sample may be a blood sample. In certain embodiments the blood sample may be a whole blood sample, a plasma sample, a serum sample, or other blood fraction sample. In certain embodiments, the methods involve the passing of the second flowthrough fraction through a third membrane with a pore size that is smaller than the second membrane pore size, wherein the second flowthrough fraction is separated into a third flowthrough fraction and a third membrane fraction, wherein the fetal microparticles are enriched in at least one of the six membrane and flowthrough fractions. In certain embodiments, the methods may involve the use of two or more membranes of decreasing pore size stacked on top of one another in order of pore size, wherein the biological sample is first passed through the membrane having the largest pore size.

In certain embodiments, the steps include passing the biological sample through a first membrane having a first membrane pore size, collecting a first flowthrough fraction and a first membrane fraction for the first membrane, passing the first flowthrough fraction through a second membrane with a pore size that is smaller than the first membrane pore size, and collecting a second flowthrough fraction and a second membrane fraction for the second membrane, wherein the fetal microparticles are enriched in at least one of the four collected flowthrough and membrane fractions. In certain embodiments, the pore size of the first and second membranes ranges from about 0.1 μm to about 1 μm. In additional aspects, the methods involve the use of a third membrane which has a pore size that is smaller than the pore size of the second membrane, to pass through the second flowthrough fraction. In other aspects, the methods involve the use of more than three membranes, wherein each membrane in the series has a pore size that is smaller than the membrane prior to that membrane in the series.

In certain other aspects, methods for enriching fetal DNA in a biological sample are provided, including passing a biological sample through a first membrane having a first membrane pore size, wherein the sample is separated into a first flowthrough fraction and a first membrane fraction; passing the first flowthrough fraction through a second membrane with a pore size that is smaller than the first membrane pore size, wherein the first flowthrough fraction is separated into a second flowthrough fraction and a second membrane fraction; and wherein the fetal microparticles are enriched in at least one of the four flowthrough and membrane fractions, and isolating DNA from the fraction enriched for the fetal microparticles, thereby enriching fetal DNA in the biological sample. In some embodiments, the biological sample may comprise at least one of a whole blood sample, plasma sample, serum sample, or other blood fraction sample. In certain embodiments, the pore size of the first and second membranes ranges from about 0.1 μm to about 1 μm. In certain embodiments, the methods involve the passing of the second flowthrough fraction through a third membrane with a pore size that is smaller than the second membrane pore size, wherein the second flowthrough fraction is separated into a third flowthrough fraction and a third membrane fraction, wherein the fetal microparticles are enriched in at least one of the six membrane and flowthrough fractions. In certain embodiments, the methods may involve the use of two or more membranes of decreasing pore size stacked on top of one another in order of pore size, wherein the biological sample is first passed through the membrane having the largest pore size.

In certain embodiments, the method comprises including passing the biological sample through a first membrane having a first membrane pore size, collecting a first flowthrough fraction and a first membrane fraction for the first membrane, passing the first flowthrough fraction through a second membrane with a pore size that is smaller than the first membrane pore size, collecting a second flowthrough fraction and a second membrane fraction for the second membrane, wherein fetal microparticles are enriched in at least one of the four collected flowthrough and membrane fractions, and isolating DNA from the fraction enriched for the fetal microparticles, thereby enriching fetal DNA in the biological sample. In certain aspects, the methods involve the use of three or more membranes, wherein each membrane in the series has a pore size that is smaller than the membrane prior to that membrane in the series. The enriched fetal DNA may be analyzed, for example, using digital PCR.

In other aspects, less invasive methods for facilitating prenatal diagnosis of a chromosomal abnormality in a fetus are provided, including the steps of passing a biological sample through a first membrane having a first membrane pore size, wherein the sample is separated into a first flowthrough fraction and a first membrane fraction; passing the first flowthrough fraction through a second membrane with a pore size that is smaller than the first membrane pore size, wherein the first flowthrough fraction is separated into a second flowthrough fraction and a second membrane fraction; and wherein the fetal microparticles are enriched in at least one of the four flowthrough and membrane fractions; isolating DNA from the fraction that is enriched for the fetal microparticles, and analyzing the DNA to detect the presence or absence of the chromosomal abnormality. In some embodiments, the biological sample may comprise at least one of a whole blood sample, plasma sample, serum sample, or other blood fraction sample. In certain embodiments, the pore size of the first and second membranes ranges from about 0.1 μm to about 1 μm. In certain embodiments, the methods involve the passing of the second flowthrough fraction through a third membrane with a pore size that is smaller than the second membrane pore size, wherein the second flowthrough fraction is separated into a third flowthrough fraction and a third membrane fraction, wherein the fetal microparticles are enriched in at least one of the six membrane and flowthrough fractions. In certain embodiments, the methods may involve the use of two or more membranes of decreasing pore size stacked on top of one another in order of pore size, wherein the biological sample is first passed through the membrane having the largest pore size.

In some embodiments the method includes the steps of obtaining a biological sample from a pregnant woman, passing the biological sample through a first membrane having a first membrane pore size, collecting a first flowthrough fraction and a first membrane fraction for the first membrane, passing the first flowthrough fraction through a second membrane with a pore size that is smaller than the first membrane pore size, collecting a second flowthrough fraction and a second membrane fraction for the second membrane, wherein fetal microparticles are enriched in at least one of the four collected flowthrough and membrane fractions, isolating DNA from the fraction that is enriched for the fetal microparticles, and analyzing the DNA to detect the presence or absence of the chromosomal abnormality. In some aspects, the methods involve the use of three or more membranes, wherein each membrane in the series has a pore size that is smaller than the membrane prior to that membrane in the series. In certain aspects, the chromosomal abnormality is an aneuploidy of chromosome 13, 18, 21, or X. In some embodiments, the less invasive methods are reliable for samples obtained from a pregnant woman when the gestational age of the fetus is less than about 16 weeks.

In certain aspects, methods for enriching disease specific microparticles (e.g., cancer microparticles) in a biological sample are provided, including the steps of passing a biological sample through a first membrane having a first membrane pore size, wherein the sample is separated into a first flowthrough fraction and a first membrane fraction; passing the first flowthrough fraction through a second membrane with a pore size that is smaller than the first membrane pore size, wherein the first flowthrough fraction is separated into a second flowthrough fraction and a second membrane fraction; and wherein the disease specific microparticles are enriched in at least one of the four flowthrough and membrane fractions. In certain embodiments, the biological sample may be a blood sample. In certain embodiments the blood sample may comprise a whole blood sample, a plasma sample, a serum sample, or other blood fraction sample. In certain embodiments, the methods involve the passing of the second flowthrough fraction through a third membrane with a pore size that is smaller than the second membrane pore size, wherein the second flowthrough fraction is separated into a third flowthrough fraction and a third membrane fraction, wherein the disease specific microparticles are enriched in at least one of the six membrane and flowthrough fractions. In certain embodiments, the methods may involve the use of two or more membranes of decreasing pore size stacked on top of one another in order of pore size, wherein the biological sample is first passed through the membrane having the largest pore size.

In certain embodiments, the steps include passing the biological sample through a first membrane having a first membrane pore size, collecting a first flowthrough fraction and a first membrane fraction for the first membrane, passing the first flowthrough fraction through a second membrane with a pore size that is smaller than the first membrane pore size, and collecting a second flowthrough fraction and a second membrane fraction for the second membrane, wherein the disease specific microparticles are enriched in at least one of the four collected flowthrough and membrane fractions. In certain embodiments, the pore size of the first and second membranes ranges from about 0.1 μm to about 1 μm. In additional aspects, the methods involve the use of a third membrane which has a pore size that is smaller than the pore size of the second membrane, to pass through the second flowthrough fraction. In other aspects, the methods involve the use of more than three membranes, wherein each membrane in the series has a pore size that is smaller than the membrane prior to that membrane in the series.

In certain other aspects, methods for enriching disease specific nucleic acids in a biological sample are provided, including passing a biological sample through a first membrane having a first membrane pore size, wherein the sample is separated into a first flowthrough fraction and a first membrane fraction; passing the first flowthrough fraction through a second membrane with a pore size that is smaller than the first membrane pore size, wherein the first flowthrough fraction is separated into a second flowthrough fraction and a second membrane fraction; and wherein disease specific microparticles are enriched in at least one of the four flowthrough and membrane fractions, and isolating DNA from the fraction enriched for the disease specific microparticles, thereby enriching disease specific DNA in the biological sample. In certain embodiments, the methods may involve the use of two or more membranes of decreasing pore size stacked on top of one another in order of pore size, wherein the biological sample is first passed through the membrane having the largest pore size.

In certain embodiments the method comprises including passing the biological sample through a first membrane having a first membrane pore size, collecting a first flowthrough fraction and a first membrane fraction for the first membrane, passing the first flowthrough fraction through a second membrane with a pore size that is smaller than the first membrane pore size, collecting a second flowthrough fraction and a second membrane fraction for the second membrane, wherein disease specific microparticles are enriched in at least one of the four collected flowthrough and membrane fractions, and isolating DNA from the fraction enriched for the disease specific microparticles, thereby enriching disease specific DNA in the biological sample. In certain aspects, the methods involve the use of three or more membranes, wherein each membrane in the series has a pore size that is smaller than the membrane prior to that membrane in the series. The enriched disease specific DNA may be analyzed, for example, using digital PCR.

In certain other aspects, the methods also may be used for detection or monitoring of a disease state. For example, methods for facilitating diagnosis of cancer or other diseases associated with cell activation, cell death, apoptosis, or circulating microparticles (or a combination thereof) are provided, including the steps of obtaining a biological sample from a patient, passing a biological sample through a first membrane having a first membrane pore size, wherein the sample is separated into a first flowthrough fraction and a first membrane fraction; passing the first flowthrough fraction through a second membrane with a pore size that is smaller than the first membrane pore size, wherein the first flowthrough fraction is separated into a second flowthrough fraction and a second membrane fraction; and wherein the disease specific microparticles are enriched in at least one of the four flowthrough and membrane fractions. In certain embodiments, the methods may involve the use of two or more membranes of decreasing pore size stacked on top of one another in order of pore size, wherein the biological sample is first passed through the membrane having the largest pore size.

In other embodiments the methods comprise the steps of passing the biological sample through a first membrane having a first membrane pore size, collecting a first flowthrough fraction and a first membrane fraction for the first membrane, passing the first flowthrough fraction through a second membrane with a pore size that is smaller than the first membrane pore size, collecting a second flowthrough fraction and a second membrane fraction for the second membrane, wherein the disease specific microparticles are enriched in at least one of the four collected flowthrough and membrane fractions, isolating DNA from the fraction that is enriched for the disease specific microparticles, and analyzing the DNA to detect the presence or absence of a mutation associated with the disease, wherein presence of the mutation indicates that the patient has the disease. In other embodiments, the methods involve the use of three or more membranes, wherein each membrane in the series has a pore size that is smaller than the membrane prior to that membrane in the series. Also provided are methods for enriching cancer or other disease specific microparticles in a biological sample using the disclosed serial size exclusion filtration methods.

### BRIEF DESCRIPTION OF THE FIGURES

Non-limiting embodiments of the methods of the invention are exemplified in the following figures.

FIG. 1 is a graph showing the percentage of fetal microparticles recovered in various fractions in a serial filtration experiment. The percentage shows the percentage of the fetal DNA as compared to the total DNA. The genome equivalents of total DNA were determined by digital PCR with primers to the β-globin gene, and the genome equivalents of fetal DNA were determined by digital PCR with primers to the Y chromosome specific sequence Y49a (DYS1) gene. The fractions are listed below each bar on the graph, including an unfiltered plasma fraction, a fraction that was captured on a 0.45 μm filter, a fraction that was captured on a 0.22 μm filter, and a fraction that was captured on a 0.10 μm filter, as well as the flowthrough (FT) fraction from each filter. The error bars are standard deviation, n=2 (i.e., the same sample was run twice).

FIG. 2 is a graph showing the level of fetal DNA enrichment in all fractions of maternal plasma collected in a serial filtration experiment. The yield is the amount of total or fetal DNA relative to the amount present in the maternal plasma prior to microparticle capture (i.e., 758.1 genomic equivalents (GE)/mL plasma and 22.90 GE/mL plasma for total and fetal DNA, respectively, before capture). The fractions of the sample are listed below each bar on the graph, including a fraction that was captured on a 0.45 μm filter, a fraction that was captured on a 0.22 μm filter, and a fraction that was captured on a 0.10 μm filter, as well as the flowthrough (FT) fractions from each filter. Fold enrichment was calculated as the percent fetal DNA found in the fraction divided by the percent fetal DNA found in initial maternal plasma. For example, 2-fold enrichment is a doubling of the fetal fraction, and 1-fold is no enrichment. The error bars are standard deviation, n=2 (i.e., the same sample was run twice).

FIG. 3 is a graph showing the fetal DNA yield from microparticles captured on each filter by pore size. The fractions are listed below each bar on the graph, including a fraction that was captured on a 0.45 μm filter, a fraction that was captured on a 0.22 μm filter, and a fraction that was captured on a 0.10 μm filter. The error bars are standard deviation, n=2 (i.e., the same sample was run twice). The percentage was calculated by dividing the percentage of fetal DNA yield after filtration by the percentage of the fetal DNA yield before filtration.

### DETAILED DESCRIPTION

Embodiments of the present invention provide methods to enrich and quantify a rare population of microparticles, cells, or nucleic acids in a complex mixture. The various embodiments of the methods involve the use of serial size exclusion filtration for the capture of a specific population of microparticles or cells and thereby, enrichment of the microparticles or cells and the nucleic acids within these microparticles or cells. The various embodiments of the methods also may involve the quantification of these nucleic acids using sensitive methods known to one of skill in the art, such as single molecule counting methods as it is expected that the amount of nucleic acids isolated will be very low, highly enriched, and may be below the detection limit for more conventional quantification methods such as spectrophotometry, dye intercalation, or quantitative PCR (qPCR)(although such conventional quantification methods may be appropriate in some instances). The disclosed enrichment methods have particular application for the isolation, enrichment, and detection of fetal DNA encapsulated in microparticles during apoptosis of placental cells. These fetal DNA-containing microparticles are known to be circulating in the maternal plasma throughout gestation. The disclosed enrichment methods also have particular application in the identification of mutations in rare disease cells (e.g., cancer cells) or disease specific microparticles (e.g., cancer microparticles) that are circulating in the blood.

### DEFINITIONS AND ABBREVIATIONS

The following terms are herein defined as they are used in this application:

The terms “microparticles,” “apoptotic bodies,” “microvesicles,” and “vesicles” are used interchangeably herein to refer to cell membrane-bound particles that may include genetic material and surface biomarkers from the cell from which they were derived, for example, during apoptosis or other type of cell death. As used herein, the term “biomarker” refers to a molecule present on or in a particular cell type (e.g., a placental alkaline phosphatase protein on the surface of fetal cells). “Fetal microparticles,” “fetal derived microparticles,” “fetal-associated microparticles,” or the like are microparticles that may be found in the bloodstream or other biological sample of an expectant mother primarily due to the apoptosis of fetal cells. Fetal microparticles may have fetal-specific biomarkers on their surfaces and contain fetal DNA. “Disease microparticles,” “disease specific microparticles,” “disease-associated microparticles,” or the like refer to microparticles that have a biomarker that is specific to a particular disease. “Cancer microparticles,” “cancer cell derived microparticles,” “cancer-associated microparticles,” or the like are microparticles that may be found in the bloodstream or other biological sample of a patient with a cancer due to the apoptosis or other type of cell death of cancer cells, or other release from cancer cells. Cancer microparticles may have tumor or cancer specific markers on their surfaces.

As used herein, the term “biological sample” encompasses any sample obtained from a biological source suitable for use in the present methods in which a rare cell, microparticle, or nucleic acid is present in the same sample with other cells, microparticles, or nucleic acids. A biological sample can, by way of non-limiting example, include whole blood, serum, plasma, other blood fraction, amniotic fluid, cultured cells, and/or chorionic villi. In certain embodiments, the biological sample is a whole blood sample, plasma sample, serum sample, any other blood fraction sample, or a combination thereof. A biological sample may be obtained from an individual by any method known to one of skill in the art, and may be obtained directly (e.g., obtaining a blood sample by venipuncture from the individual) or indirectly (e.g., obtaining a biological sample from a healthcare provider, hospital, or practitioner that directly obtained the biological sample from the patient).

As used herein, the term “subject” is used to refer to a human or any non-human animal (e.g., mouse, rat, rabbit, dog, cat, cattle, swine, sheep, horse, or primate). Preferably, the subject is human. A subject can be a “patient,” which refers to a human presenting to a medical provider for diagnosis, treatment, or care for a condition or disease. The terms “patient” and “individual” may be used interchangeably herein. In one embodiment, the patient or individual is a woman and her condition is that she is pregnant. In some embodiments, a subject can be afflicted with or susceptible to a disease or disorder but may or may not display symptoms of the disease or disorder.

As used herein, the term “apoptosis” refers to a form of programmed cell death. Apoptosis causes morphological changes to the surface of a cell, often resulting in “blebbing” of the cell membrane, which causes microparticles to form. Because the microparticles are formed from the cell membrane, they carry any membrane-specific markers that the original cells also expressed (e.g., fetal-specific markers, disease-specific markers, or tumor-specific markers). In one example, apoptosis occurs naturally to placental or fetal cells during a pregnancy.

The term “pore size” is used herein to refer to the diameter of the pores or holes in a membrane. As used herein, the terms “membrane” or “filter” refers to a membrane with pores of relatively uniform size that are made, for example, of a material such as polyethersulfone (PES) or polyvinylidene fluoride (PVDF). Other materials, including, but not limited to, nitrocellulose, regenerated cellulose, polypropylene, nylon, and mixed cellulose esters (MCE) also may be used for the membranes. The membranes allow molecules of a certain size to pass through the membrane, and molecules of a larger size are captured on the membrane. The term “membrane fraction” or “filter fraction” refers to the fraction that is captured on a membrane, and the “flowthrough” or “flowthrough fraction” refers to the fraction of the sample that passes through the membrane. As used herein, the phrase “serial size exclusion filtration” refers to a method in which a mixture is passed through a series of at least two filters with decreasing pore size. In some embodiments, the mixture is passed through a series of at least three filters with decreasing pore size. In other embodiments, the mixture is passed through a series of four or more filters with decreasing pore size.

The term “enrichment” is used herein to refer to the concentration of a rare microparticle, cell, or nucleic acid in a complex mixture (e.g., the enrichment of a fetal microparticle in a maternal blood sample). Enrichment is determined by comparing the ratio of the amount of target material (e.g., a fetal microparticle) to other material in the sample after filtration has taken place, to the ratio of the target material to other material in the initial sample before filtration. Enrichment results in an increase in the quality of the filtered material with respect to detecting the target material (i.e., an increase in the ratio of target material to other material present).

The term “chromosomal abnormality” is used herein to refer to any kind of defect associated with a chromosome, including single or multiple base pair deletions, additions, and substitutions; translocations; or defects in the numbers of complete chromosomes or sets of chromosomes. The term “aneuploidy” refers to when one or more chromosomes are missing or are present in more than the normal number of copies. Aneuploidy is associated with many diseases or syndromes, including, but not limited to, Down syndrome, Edwards syndrome, Patau syndrome, and Turner syndrome.

“Polymerase chain reaction” or “PCR” refers to a molecular biology technique used to amplify (increase the concentration of) and/or quantify a small amount of nucleic acids (e.g., DNA). There are many forms of PCR, such as digital PCR or real time PCR, that are specialized for a particular purpose. For example, digital PCR is a refinement of the original PCR technique that is better able to provide absolute quantification of nucleic acids by partitioning individual nucleic acid molecules in separate regions. Various other PCR techniques, including those described herein (e.g., quantitative real time PCR, emulsion PCR, multiplex PCR, and digital PCR), are well known by those skilled in the art and may be used in the present methods depending upon the amount of nucleic acids present in a particular sample.

### Enrichment Methods for Fetal Microparticles in a Complex Composition

Certain embodiments of the present invention provide the use of size exclusion via filtration to enrich for vesicles that contain nucleic acids from a complex mixture such as blood. Microparticles containing nucleic acids have been reported to range in size from about 0.1 μm to about 1 μm. Based on their wide range of sizes, these microparticles can be selectively captured on filters of various diameter pores. Once captured, the microparticles can be solubilized, and the nucleic acids can be purified directly from the membrane using standard molecular biological methods.

The capture of microparticles by size filtration provides a simple, fast, and cost-effective method for the enrichment of fetal nucleic acids from maternal plasma. Once the fetal nucleic acids are enriched, the fetal nucleic acids can be examined using known molecular biology techniques such as real time PCR or digital PCR to determine detailed genetic information about the fetus.

Methods for enriching fetal microparticles in a biological sample are provided, including the steps of passing a biological sample through a first membrane having a first membrane pore size, wherein the sample is separated into a first flowthrough fraction and a first membrane fraction; passing the first flowthrough fraction through a second membrane with a pore size that is smaller than the first membrane pore size, wherein the first flowthrough fraction is separated into a second flowthrough fraction and a second membrane fraction; and wherein the fetal microparticles are enriched in at least one of the four fractions. The biological sample may comprise at least one of a whole blood sample, a plasma sample, a serum sample, or any other blood fraction sample, and the sample may be obtained from the patient by any method known to one of skill in the art. Various methods for separating a whole blood sample into two or more blood fraction samples are well known to one of skill in the art. In one embodiment, a whole blood sample is obtained by venipuncture from an individual and then centrifuged using low speed centrifugation in order to separate the plasma fraction from the rest of the blood fractions.

The biological sample is passed through a series membranes, wherein each of the membranes has relatively uniform pore size throughout the membrane. In some embodiments, the membranes are made of a material such as polyethersulfone (PES) or polyvinylidene fluoride (PVDF). In other embodiments, the membranes are composed of other materials such as, but not limited to, nitrocellulose, regenerated cellulose, polypropylene, nylon, and mixed cellulose esters (MCE). In certain embodiments, the methods involve at least 2 membranes. In other embodiments, the methods involve at least three membranes. In still other embodiments, the methods involve four or more membranes. Because microparticles vary widely in size (though they are typically between about 0.1 and about 1 μm) and because the quantity of fetal microparticles in maternal plasma is unpredictable between individuals and throughout gestation, the use of a series of filters of decreasing pore sizes allows for the capture of as many microparticles as possible. Therefore, in certain embodiments, the pore sizes of the membranes range from about 0.1 μm to about 1 μm. In certain other embodiments, the pore sizes of the membranes range from about 0.025 μm to about 5 μm from about 0.025 μm to about 4 μm, about 0.025 μm to about 3 μm, about 0.05 μm to about 3 μm, about 0.05 μm to about 2 μm, about 0.1 μm to about 2 μm, about 0.05 μm to about 1 μm, about 0.1 μm to about 0.5 μm, or from about 0.1 μm to about 1 μm. In some embodiments, the pore sizes vary within about a 50% range of each size.

In one specific embodiment, the methods include a first membrane with a pore diameter that is about 0.45 μm, and a second membrane with a pore diameter that is about 0.22 μm (or other pore sizes within about a 50% range of each size). Fractions may be collected for both the material that is retained by each filter (membrane fraction), and the material that passes through each filter (flowthrough fraction). The microparticles become enriched in at least one of the filter or flowthrough fractions. In additional aspects, the methods involve the use of a third membrane which has a pore size that is smaller than the pore size of the second membrane, to pass through the second flowthrough fraction. For example, the methods may include three membranes with pore diameters that are about 0.45 μm, about 0.22 μm, and about 0.1 μm, respectively (or other pore sizes within about a 50% range of each size). Similarly, the methods also may involve the use of a fourth membrane or additional membranes, each of which has a pore size that is smaller than the previous membrane in the series.

In certain embodiments, the methods may involve the use of two or more membranes of decreasing pore size stacked on top of one another in order of pore size, wherein the biological sample is first passed through the membrane having the largest pore size. The microparticles become enriched in at least one of the filter fractions or in the flowthrough fraction.

In certain embodiments, the desired population of microparticles is further enriched prior to or after filtration. For example, counterstains such as DAPI, propidium iodide, Hoechst, or other another stain known to those of skill in the art that also binds to nucleic acids under specific cellular conditions can be used to further subfractionate and enrich for those microparticles that contain nucleic acids. In other embodiments, the biological sample is first selectively depleted of maternal microparticles by using binding molecules specific for a maternal biomarker.

### Methods for Enriching Fetal DNA in a Complex Composition

Also provided are methods for enriching fetal DNA in a biological sample, including passing a biological sample through a first membrane having a first membrane pore size, wherein the sample is separated into a first flowthrough fraction and a first membrane fraction; passing the first flowthrough fraction through a second membrane with a pore size that is smaller than the first membrane pore size, wherein the first flowthrough fraction is separated into a second flowthrough fraction and a second membrane fraction; and wherein the fetal microparticles are enriched in at least one of the four membrane and flowthrough fractions; and isolating nucleic acids from the fraction enriched for the fetal microparticles, thereby enriching fetal nucleic acids in the biological sample.

The biological sample may comprise at least one of a whole blood sample, a plasma sample, a serum sample, or any other blood fraction, and the sample may be obtained from the patient by any method known to one of skill in the art. The biological sample is passed through a series of membranes, such as a PES or PVDF membrane. In other embodiments, the membranes are composed of other materials such as, but not limited to, nitrocellulose, regenerate cellulose, polypropylene, or PTFC. In certain embodiments, the methods involve at least two membranes. In other embodiments, the methods involve at least three membranes. In still other embodiments, the methods involve four or more membranes.

Because microparticles vary widely in size and the quantity of fetal microparticles in maternal plasma is unpredictable between individuals and throughout gestation, the use of a series of filters of decreasing pore sizes allows for the capture of as many microparticles as possible. Therefore, in certain embodiments, the pore sizes of the membranes range from about 0.1 μm to about 1 μm. In certain other embodiments, the pore sizes of the membranes range from about 0.025 μm to about 5 μm from about 0.025 μm to about 4 μm, about 0.025 μm to about 3 μm, about 0.05 μm to about 3 μm, about 0.05 μm to about 2 μm, about 0.1 μm to about 2 μm, about 0.05 μm to about 1 μm, about 0.1 μm to about 0.5 μm, or from about 0.1 μm to about 1 μm. In some embodiments, the pore sizes vary within about a 50% range of each size.

In one specific embodiment, the methods include a first membrane with a pore diameter that is about 0.45 μm, and a second membrane with a pore diameter that is about 0.22 μm (or other pore sizes within about a 50% range of each size). Samples are collected for both the material that is retained by each filter, and the material that passes through each filter (flowthrough). The microparticles become enriched in at least one of the filter or flowthrough fractions. In additional aspects, the methods involve the use of a third membrane which has a pore size that is smaller than the pore size of the second membrane, to pass through the second flowthrough fraction. For example, the methods may include three membranes with pore diameters that are about 0.45 μm, about 0.22 μm, and about 0.1 μm, respectively (or other pore sizes within about a 50% range of each of these sizes). Similarly, the methods also may involve the use of a fourth membrane or additional membranes, each of which has a pore size that is smaller than the previous membrane in the series.

In certain embodiments, the methods may involve the use of two or more membranes of decreasing pore size stacked on top of one another in order of pore size, wherein the biological sample is first passed through the membrane having the largest pore size. The microparticles become enriched in at least one of the filter fractions or in the flowthrough fraction.

In certain embodiments, the desired population of microparticles is further enriched prior to or after filtration. For example, counterstains such as DAPI, propidium iodide, Hoechst, or other another stain known to those of skill in the art that also binds to nucleic acids under specific cellular conditions can be used to further subfractionate and enrich for those microparticles that contain nucleic acids. In other embodiments, the biological sample is first selectively depleted of maternal microparticles by using binding molecules specific for a maternal biomarker.

Any trapped microparticles on the filters can be solubilized by molecular biology methods known to one of skill in the art. Examples of such methods include the use of detergents or chaotropic salts in order to solubilize or disaggregate the microparticles. Fetal nucleic acids (e.g., DNA) may be then extracted from the filter and flowthrough fractions using standard molecular biology methods known to one of skill in the art. The extraction can be carried out either directly in the housing that holds the filter without removing the membrane from the assembly, or after separating the filters from the housing. One example of a DNA extraction method is the method used with the QIAAMP Circulating Nucleic Acid kit (Qiagen). Alternatively, in some instances, the sample could be incubated with Proteinase K for 30 minutes at 56° C. while shaking at 400 rpm, followed by heat inactivation at 95° C. for 20 minutes, centrifugation at 5,000 g for 5 minutes, and removal of the supernatant from the debris for further analysis. Various modifications may also be suitable for extraction in some embodiments. In addition, other suitable methods for DNA extraction are well known to one of skill in the art.

Fetal nucleic acid quantities can be determined in each fraction by a sensitive method such as real-time PCR or digital PCR. The fetal nucleic acids may then also be examined for any genetic defects or chromosomal abnormalities. In some embodiments, multiplex PCR may be used (i.e., more than one fetal gene may be amplified simultaneously in a single PCR reaction). Alternatively, the fetal nucleic acids may be analyzed by sequencing methods known to one of skill in the art. Other methods by which the target molecules may be amplified include, but are not limited to whole genome amplification, strand displacement amplification, rolling circle amplification, ligase chain amplification, and multiple PCR methods including quantitative real time PCR, emulsion PCR, and digital PCR. The amplified targets may be detected with methods such as, but not limited to fluorescence such as a probe, dye, or nucleotide; chemiluminescence; radioactivity; capillary electrophoresis; microarrays; sequencing; mass spectrometry; and nanostring technology. The disclosed enrichment methods may be performed as early as the first trimester of the pregnancy, and may be repeated throughout the pregnancy to continue to monitor the health of the developing fetus.

### Less Invasive Methods for Fetal Facilitating Prenatal Diagnosis

Less invasive methods for facilitating prenatal diagnosis of a chromosomal abnormality in a fetus are provided, including the steps of obtaining a biological sample from a pregnant woman, passing a biological sample through a first membrane having a first membrane pore size, wherein the sample is separated into a first flowthrough fraction and a first membrane fraction; passing the first flowthrough fraction through a second membrane with a pore size that is smaller than the first membrane pore size, wherein the first flowthrough fraction is separated into a second flowthrough fraction and a second membrane fraction; and wherein the fetal microparticles are enriched in at least one of the four membrane and flowthrough fractions; isolating nucleic acids from the fraction that is enriched for the fetal microparticles, and analyzing the nucleic acids to detect the presence or absence of the chromosomal abnormality.

In one embodiment, the chromosomal abnormality is a mutation that is associated with a disease. In certain aspects, the chromosomal abnormality may be an aneuploidy of chromosome 13, 18, 21, or X. In certain other aspects, the chromosomal abnormality is a paternally controlled allele. In certain other aspects, the chromosomal abnormality is a point mutation. In some embodiments, the less invasive methods are reliable for samples obtained from a pregnant woman when the gestational age of the fetus is less than about 16 weeks. In one embodiment, the noninvasive methods are reliable for samples obtained from a pregnant woman during her first trimester of pregnancy.

The biological sample may comprise at least one of a whole blood sample, plasma sample, serum sample, or any other blood fraction sample, and the sample may be obtained from the patient by any method known to one of skill in the art. The biological sample is passed through a series of membranes, such as a PES or PVDF membrane. In other embodiments, the membranes are composed of other materials such as, but not limited to, nitrocellulose, regenerated cellulose, polypropylene, nylon, and mixed cellulose esters (MCE). In certain embodiments, the methods involve at least two membranes. In other embodiments, the methods involve at least three membranes. In still other embodiments, the methods involve four or more membranes. Because microparticles vary widely in size and the quantity of fetal microparticles in maternal plasma is unpredictable between individuals and throughout gestation, the use of a series of filters of decreasing pore sizes allows for the capture of as many microparticles as possible. Therefore, in certain embodiments, the pore sizes of the membranes range from about 0.1 μm to about 1 μm. In certain other embodiments, the pore sizes of the membranes range from about 0.025 μm to about 5 μm from about 0.025 μm to about 4 μm, about 0.025 μm to about 3 μm, about 0.05 μm to about 3 μm, about 0.05 μm to about 2 μm, about 0.1 μm to about 2 μm, about 0.05 μm to about 1 μm, about 0.1 μm to about 0.5 μm, or from about 0.1 μm to about 1 μm. In some embodiments, the pore sizes vary within about a 50% range of each size.

In one specific embodiment, the methods include a first membrane with a pore diameter that is about 0.45 μm, and a second membrane with a pore diameter that is about 0.22 μm (or other pore sizes within about a 50% range of each size). Samples are collected for both the material that is retained by each filter, and the material that passes through each filter (flowthrough). The microparticles become enriched in at least one of the filter or flowthrough fractions. In additional aspects, the methods involve the use of a third membrane which has a pore size that is smaller than the pore size of the second membrane, to pass through the second flowthrough fraction. For example, the methods may include three membranes with pore diameters that are about 0.45 μm, about 0.22 μm, and about 0.1 μm, respectively (or other pore sizes within about a 50% range of each size). Similarly, the methods also may involve the use of a fourth membrane or additional membranes, each of which has a pore size that is smaller than the previous membrane in the series.

In certain embodiments, the methods may involve the use of two or more membranes of decreasing pore size stacked on top of one another in order of pore size, wherein the biological sample is first passed through the membrane having the largest pore size. The microparticles become enriched in at least one of the filter fractions or in the flowthrough fraction.

In certain embodiments, the desired population of microparticles is further enriched prior to or after filtration. For example, counterstains such as DAPI, propidium iodide, Hoechst, or other another stain known to those of skill in the art that also binds to nucleic acids under specific cellular conditions can be used to further subfractionate and enrich for those microparticles that contain nucleic acids. In other embodiments, the biological sample is first selectively depleted of maternal microparticles by using binding molecules specific for a maternal biomarker.

Any trapped microparticles on the filters can be solubilized, and fetal nucleic acids may be extracted from the filter and flowthrough fractions using standard molecular biology methods known to one of skill in the art. The extraction can be carried out either directly in the housing that holds the filter without removing the membrane from the assembly, or after separating the filters from the housing. Fetal nucleic acids quantities can be determined in each fraction by a sensitive method such as real-time PCR or digital PCR. The fetal nucleic acids may then also be examined for any genetic defects or chromosomal abnormalities. In some embodiments, multiplex PCR may be used (i.e., more than one fetal gene may be amplified simultaneously in a single PCR reaction). Alternatively, the fetal nucleic acids may be analyzed by sequencing methods known to one of skill in the art. Other methods by which the target molecules may be amplified include, but are not limited to whole genome amplification, strand displacement amplification, rolling circle amplification, ligase chain amplification, and multiple PCR methods including quantitative real time PCR, emulsion PCR, and digital PCR. The amplified targets may be detected with methods such as, but not limited to fluorescence such as a probe, dye, or nucleotide; chemiluminescence; radioactivity; capillary electrophoresis; microarrays; sequencing; mass spectrometry; and nanostring technology. The disclosed enrichment methods may be performed as early as the first trimester of the pregnancy, and may be repeated throughout the pregnancy to continue to monitor the health of the developing fetus.

### Enrichment Methods for Disease Specific Microparticles in a Complex Composition

The disclosed methods also can be applied to the detection of microparticles specific to diseases associated with cell activation, cell death, apoptosis, or other release of disease specific microparticles (e.g., cancer). For example, methods for enriching cancer microparticles or other disease specific microparticles in a complex mixture are provided, as well as methods for facilitating diagnosis of or monitoring progression of cancer or other diseases associated with cell death and apoptosis, using serial size exclusion filtration.

Certain embodiments of the present invention provide methods for facilitating diagnosis of or monitoring the progression of cancer or other disease, including the steps of obtaining a biological sample from a patient, passing a biological sample through a first membrane having a first membrane pore size, wherein the sample is separated into a first flowthrough fraction and a first membrane fraction; passing the first flowthrough fraction through a second membrane with a pore size that is smaller than the first membrane pore size, wherein the first flowthrough fraction is separated into a second flowthrough fraction and a second membrane fraction; and wherein the disease microparticles are enriched in at least one of the four membrane and flowthrough fractions; isolating nucleic acids from the fraction that is enriched for the disease microparticles, and analyzing the nucleic acids to detect the presence or absence of a mutation associated with the disease, wherein presence of the mutation indicates that the patient has the disease.

The biological sample may comprise at least one of a blood sample, plasma sample, other blood fraction sample, or sample of any bodily fluid that has come in contact with cancer or disease cells (e.g., bile, urine, mucus, cerebrospinal fluid, peritoneal fluid, lymphatic fluid, etc.). The biological sample is passed through a series of membranes, such as a PES or PVDF membrane. In other embodiments, the membranes are composed of other materials such as, but not limited to, nitrocellulose, regenerated cellulose, polypropylene, nylon, and mixed cellulose esters (MCE). In certain embodiments, the methods involve at least two membranes. In other embodiments, the methods involve at least three membranes. In still other embodiments, the methods involve four or more membranes. Because microparticles vary widely in size, the use of a series of filters of decreasing pore sizes allows for the capture of as many microparticles as possible. Therefore, in certain embodiments, the pore sizes of the membranes range from about 0.1 μm to about 1 μm. In certain other embodiments, the pore sizes of the membranes range from about 0.025 μm to about 5 μm from about 0.025 μm to about 4 μm, about 0.025 μm to about 3 μm, about 0.05 μm to about 3 μm, about 0.05 μm to about 2 μm, about 0.1 μm to about 2 μm, about 0.05 μm to about 1 μm, about 0.1 μm to about 0.5 μm, or from about 0.1 μm to about 1 μm. In some embodiments, the pore sizes vary within about a 50% range of each size.

In one specific embodiment, the methods include a first membrane with a pore diameter that is about 0.45 μm, and a second membrane with a pore diameter that is about 0.22 μm (or other pore sizes within about a 50% range of each size). Samples are collected for both the material that is retained by each filter, and the material that passes through each filter (flowthrough). The microparticles become enriched in at least one of the filter or flowthrough fractions. In additional aspects, the methods involve the use of a third membrane which has a pore size that is smaller than the pore size of the second membrane, to pass through the second flowthrough fraction. For example, the methods may include three membranes with pore diameters that are about 0.45 μm, about 0.22 μm, and about 0.1 μm, respectively (or other pore sizes within about a 50% range of each size). Similarly, the methods also may involve the use of a fourth membrane or additional membranes, each of which has a pore size that is smaller than the previous membrane in the series.

In certain embodiments, the methods may involve the use of two or more membranes of decreasing pore size stacked on top of one another in order of pore size, wherein the biological sample is first passed through the membrane having the largest pore size. The microparticles become enriched in at least one of the filter fractions or in the flowthrough fraction.

In certain embodiments, the desired population of microparticles is further enriched prior to or after filtration. For example, counterstains such as DAPI, propidium iodide, Hoechst, or other another stain known to those of skill in the art that also binds to nucleic acids under specific cellular conditions can be used to further subfractionate and enrich for those microparticles that contain nucleic acids. In other embodiments, the biological sample is first selectively depleted of microparticles produced by a cell type that would be expected in the particular biological sample, by using binding molecules specific for a biomarker present on those cells.

Any trapped microparticles on the filters can be solubilized, and DNA may be extracted from the filter and flowthrough fractions using standard molecular biology methods known to one of skill in the art. The extraction can be carried out either directly in the housing that holds the filter without removing the membrane from the assembly, or after separating the filters from the housing. DNA quantities can be determined in each fraction by a sensitive method such as real-time PCR or digital PCR, and the DNA may be examined for any mutation specific for the cancer or other disease. In some embodiments, multiplex PCR may be used (i.e., more than one gene may be amplified simultaneously in a single PCR reaction). Alternatively, the DNA may be analyzed by sequencing methods known to one of skill in the art. Other methods by which the target molecules may be amplified include, but are not limited to whole genome amplification, strand displacement amplification, rolling circle amplification, ligase chain amplification, and multiple PCR methods including quantitative real time PCR, emulsion PCR, and digital PCR. The amplified targets may be detected with methods such as, but not limited to fluorescence such as a probe, dye, or nucleotide; chemiluminescence; radioactivity; capillary electrophoresis; microarrays; sequencing; mass spectrometry; and nanostring technology.

It should be understood that the foregoing relates to certain embodiments of the invention and that numerous changes may be made therein without departing from the scope of the invention. The invention is further illustrated by the following examples, which are not to be construed in any way as imposing limitations upon the scope thereof. On the contrary, it is to be clearly understood that resort may be had to various other embodiments, modifications, and equivalents thereof, which, after reading the description herein may suggest themselves to those skilled in the art without departing from the spirit of the present invention and/or the scope the appended claims.

### EXAMPLES

The present invention may be better understood by reference to the following non-limiting examples.

### Example 1

### Enrichment of Fetal DNA in Maternal Blood Sample

A whole blood sample was drawn from a pregnant woman carrying a male fetus at 24 weeks gestation. The blood sample was centrifuged at 1600 g for 10 minutes at room temperature to separate the plasma fraction. The plasma sample was removed to a new tube and centrifuged an additional 10 minutes at 4200 g at room temperature to remove cellular debris and platelets. Next, the plasma was removed from the tube, and one mL of the plasma was saved as the pre-filtration fraction. The remainder of the plasma sample was serially passed through 0.45 μm, 0.22 μm, and 0.10 μm pore-sized PES filters. First, the plasma sample was passed through a 0.45 μm filter at 2-3 drops/second. One mL of the flowthrough from that filter size was saved, and the remainder was then passed through a 0.22 μm filter. One mL of the flowthrough from that filter size was saved, and the remainder was then passed through a 0.1 μm filter. Phosphate buffered saline (PBS) buffer was added to each of the filters to wash any non-specific materials off of the filters, creating the 0.45 μm, 0.22 μm, and 0.10 μm fractions, respectively.

All fractions, including the flowthrough and captured microparticles on the filters, were extracted for DNA using the QIAAMP Circulating Nucleic Acid kit (Qiagen, Valencia, Calif.). Quantitative real-time PCR was then used to quantify the DNA recovery and enrichment. The results of this serial filtration experiment are shown in FIGS. 1, 2, and 3.

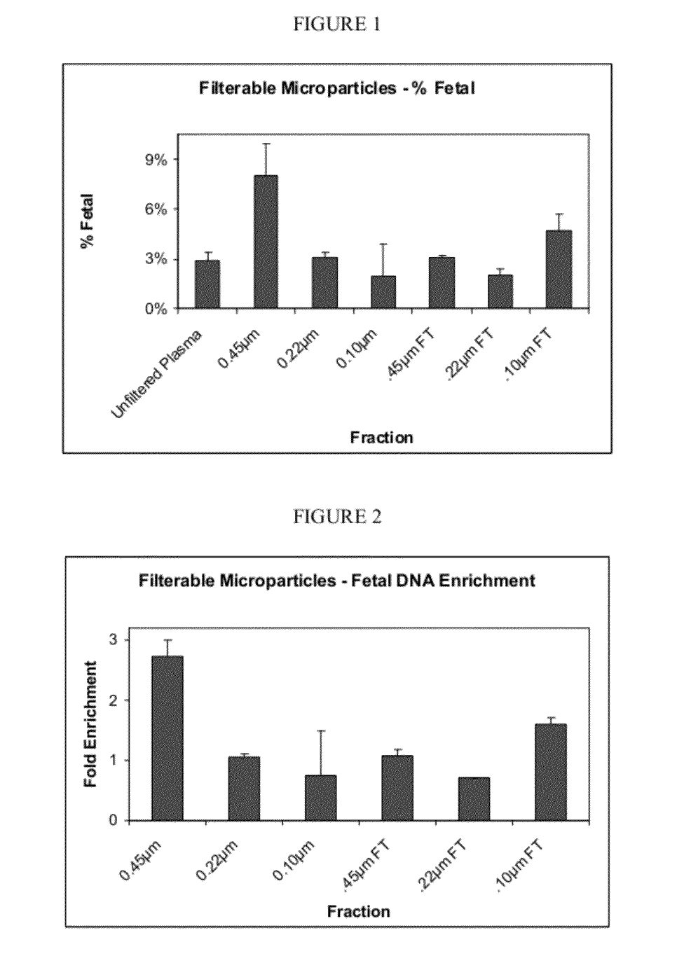

FIG. 1 shows the percentage of fetal microparticles recovered in various fractions (i.e., an unfiltered plasma fraction, a fraction that was captured on the 0.45 μm filter, a fraction that was captured on the 0.22 μm filter, and a fraction that was captured on the 0.10 μm filter, as well as the flowthrough fractions from each filter). The percentage is of the fetal DNA as compared to the total DNA. The genome equivalents of total DNA were determined by digital PCR with primers to the β-globin gene, and the genome equivalents of fetal DNA were determined by digital PCR with primers to the DYS1 gene. The error bars are standard deviation, n=2. FIG. 2 shows the level of fetal DNA enrichment in each of the fractions. Fold enrichment was calculated as the percent fetal DNA found in the fraction divided by the percent fetal DNA found in initial maternal plasma. For example, 2-fold enrichment is a doubling of the fetal fraction, and 1-fold is no enrichment. The error bars are standard deviation, n=2. The results showed that an approximately 3-fold enrichment of the fetal DNA was achieved using this serial filtration method.

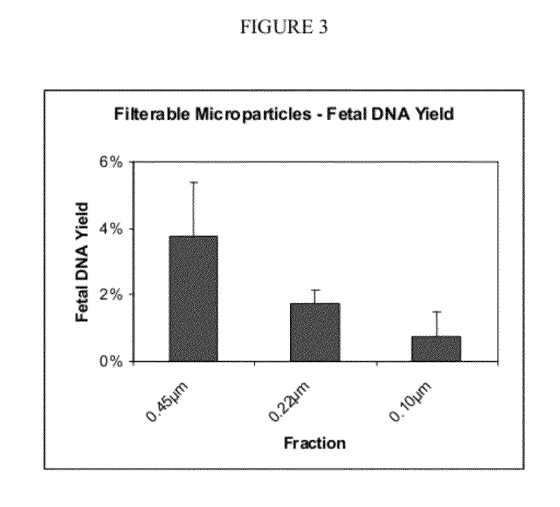

FIG. 3 is a graph showing the fetal DNA yield from microparticles captured on each of the three filters. The fractions are listed below each bar on the graph, including the fraction that was captured on the 0.45 μm filter, the fraction that was captured on the 0.22 μm filter, and the fraction that was captured on the 0.10 μm filter. The error bars are standard deviation, n=2. The fetal yield ranged from approximately 1% to approximately 4%. The percentage was calculated by dividing the percentage of fetal DNA yield after filtration by the percentage of the fetal DNA yield before filtration. In this example, enrichment was primarily achieved with the 0.45 μm filter, but it is expected that samples from different individuals and at a range of gestational ages will vary in their microparticle quantity and size. Therefore, it is recommended that serial size exclusion filtration be performed in each enrichment experiment to ensure that the fetal microparticles are enriched in at least one of the samples.

### Example 2

### Prenatal Diagnosis of a Fetal Chromosomal Abnormality

An expectant mother with a family history of Down syndrome wishes to know whether her 12-week old fetus has the disease. A whole blood sample is obtained from the patient at 12 weeks gestation and centrifuged at 1600 g for 10 minutes at room temperature to separate the plasma fraction. The plasma fraction is then spun an additional 10 minutes at 4200 g and room temperature to remove cellular debris and platelets. Next, the plasma is serially passed through 0.45 μm, 0.22 μm, and 0.10 μm pore-sized PES filters. All fractions, including the flowthrough and captured microparticles on the filters, are extracted for DNA using the QCNA procedure (Qiagen, Valencia, Calif.). The results show an approximate 2-fold enrichment of the fetal fraction is achieved with one of the filters. The DNA is characterized using standard molecular biology techniques to detect aneuploidy or other specific chromosomal abnormalities. It is determined that the fetus has only two copies of chromosome 21 and thus does not have Downs syndrome. This information is provided to the patient during the first trimester.

### Example 3

### Enrichment of Cancer Microparticles in Blood Sample

A whole blood sample is obtained from a patient suspected of having a lymphoma. The whole blood sample is centrifuged at 1600 g for 10 minutes at room temperature to separate the plasma fraction. The plasma fraction is then spun an additional 10 minutes at 4200 g and room temperature to remove cellular debris and platelets. Next, the plasma is serially passed through 0.45 μm, 0.22 μm, and 0.10 μm pore-sized PES filters. All fractions, including the flowthrough and captured microparticles on the filters, are extracted for DNA using the QCNA procedure. The results show an approximate 3-fold enrichment of the fetal fraction is achieved with one of the filters. The DNA is characterized using standard molecular biology techniques to detect a mutation associated with the lymphoma. The relevant mutation is detected, and this information is provided to the patient along with proposed treatment options.

While the invention has been described and illustrated with reference to certain embodiments thereof, those skilled in the art will appreciate that various changes, modifications and substitutions can be made therein without departing from the spirit and scope of the invention. All patents, published patent applications, and other non-patent references referred to herein are incorporated by reference in their entireties.

### REFERENCES

Orozco A F, Jorgez C J, Horne C, Marquez-Do D A, Chapman M R, Rodgers J R, Bischoff F Z, Lewis D E (2008) Membrane protected apoptotic trophoblast microparticles contain nucleic acids: relevance to preeclampsia. The American Journal of Pathology 173(6):1595-608
Orozco A F, Lewis D E (2010) Flow cytometric analysis of circulating microparticles in plasma. Cytometry Part A 77(6):502-14.
Current Protocols in Molecular Biology, Ausubel F M, Brent R, Kingston R E, Moore D D, Seidman J G, Smith J A, Struhl K, editors (John Wiley & Sons, Inc.)

1. A method for enriching fetal microparticles in a biological sample, comprising:
passing the biological sample through a first membrane having a first membrane pore size, wherein the sample is separated into a first flowthrough fraction and a first membrane fraction;
passing the first flowthrough fraction through a second membrane with a pore size that is smaller than the first membrane pore size, wherein the first flowthrough fraction is separated into a second flowthrough fraction and a second membrane fraction, and wherein the fetal microparticles are enriched in at least one of the four membrane and flowthrough fractions.

2. The method of claim 1, wherein the biological sample is a whole blood sample, plasma sample, serum sample, or any other blood fraction sample.

3. The method of claim 1, wherein the pore size of the first and second membranes ranges from about 0.1 μm to about 1 μm.

4. The method of claim 1, wherein the pore size of the first membrane is about 0.45 μm, and the pore size of the second membrane is about 0.22 μm.

5. The method of claim 1, wherein the first and second membranes are stacked, and wherein the biological sample is passed through the stack.

6. The method of claim 1, further comprising:
passing the second flowthrough fraction through a third membrane with a pore size that is smaller than the second membrane pore size, wherein the second flowthrough fraction is separated into a third flowthrough fraction and a third membrane fraction; and
wherein the fetal microparticles are enriched in at least one of the six membrane and flowthrough fractions.

7. The method of claim 6, wherein the pore size of the first membrane is about 0.45 μm, the pore size of the second membrane is about 0.22 μm, and the pore size of the third membrane is about 0.1 μm.

8. A method for enriching fetal DNA in a biological sample, comprising
passing the biological sample through a first membrane having a first membrane pore size, wherein the sample is separated into a first flowthrough fraction and a first membrane fraction;
passing the first flowthrough fraction through a second membrane with a pore size that is smaller than the first membrane pore size, wherein the first flowthrough fraction is separated into a second flowthrough fraction and a second membrane fraction, and wherein fetal microparticles are enriched in at least one of the four membrane and flowthrough fractions, and
isolating DNA from the fraction enriched for the fetal microparticles, thereby enriching fetal DNA in the biological sample.

9. The method of claim 8, wherein the pore size of the first and second membranes ranges from about 0.1 μm to about 1 μm.

10. The method of claim 8, wherein the first and second membranes are stacked, and wherein the biological sample is passed through the stack.

11. The method of claim 8, further comprising:
passing the second flowthrough fraction through a third membrane with a pore size that is smaller than the second membrane pore size, wherein the second flowthrough fraction is separated into a third flowthrough fraction and a third membrane fraction;
and wherein the fetal microparticles are enriched in at least one of the six membrane and flowthrough fractions.

12. A method for facilitating prenatal diagnosis of a chromosomal abnormality in a fetus, comprising
obtaining a biological sample from a pregnant woman,
passing the biological sample through a first membrane having a first membrane pore size, wherein the sample is separated into a first flowthrough fraction and a first membrane fraction;
passing the first flowthrough fraction through a second membrane with a pore size that is smaller than the first membrane pore size, wherein the first flowthrough fraction is separated into a second flowthrough fraction and a second membrane fraction, and wherein fetal microparticles are enriched in at least one of the four membrane and flowthrough fractions;
isolating DNA from the fraction that is enriched for the fetal microparticles; and
analyzing the DNA to detect the presence or absence of the chromosomal abnormality.

13. The method of claim 12, wherein the biological sample is a whole blood sample, a plasma sample, a serum sample, or any other blood fraction sample.

14. The method of claim 12, wherein the pore size of the first and second membranes ranges from about 0.1 μm to about 1 μm.

15. The method of claim 12, wherein the first and second membranes are stacked, and wherein the biological sample is passed through the stack.

16. The method of claim 12, further comprising:
passing the second flowthrough fraction through a third membrane with a pore size that is smaller than the second membrane pore size, wherein the second flowthrough fraction is separated into a third flowthrough fraction and a third membrane fraction; and
wherein the fetal microparticles are enriched in at least one of the six membrane and flowthrough fractions.

17. The method of claim 12, wherein the chromosomal abnormality is an aneuploidy.

18. The method of claim 12, wherein the chromosomal abnormality is a mutation associated with a disease.

19. The method of claim 12, wherein the biological sample is obtained from the woman when the gestational age of the fetus is less than about 16 weeks.

20. The method of claim 12, wherein the DNA is analyzed using digital PCR.

21. A method for facilitating diagnosis of cancer in an individual, comprising:
obtaining a biological sample from the individual,
passing the biological sample through a first membrane having a first membrane pore size, wherein the sample is separated into a first flowthrough fraction and a first membrane fraction;
passing the first flowthrough fraction through a second membrane with a pore size that is smaller than the first membrane pore size, wherein the first flowthrough fraction is separated into a second flowthrough fraction and a second membrane fraction, and wherein cancer microparticles are enriched in at least one of the four membrane and flowthrough fractions;
isolating DNA from the fraction that is enriched for the cancer microparticles; and
analyzing the DNA to detect the presence or absence of a mutation associated with cancer, wherein presence of the mutation indicates that the patient has cancer.

22. The method of claim 21, wherein the biological sample is a blood sample, plasma sample, serum sample, other blood fraction sample, or a sample of a bodily fluid that was in contact with cancer cells.

23. The method of claim 21, wherein the pore size of the first and second membranes ranges from about 0.1 μm to about 1 μm.

24. The method of claim 23, wherein the pore size of the first membrane is about 0.45 μm, and the pore size of the second membrane is about 0.22 μm.

25. The method of claim 21, wherein the first and second membranes are stacked, and wherein the biological sample is passed through the stack.

26. The method of claim 21, further comprising:
passing the second flowthrough fraction through a third membrane with a pore size that is smaller than the second membrane pore size, wherein the second flowthrough fraction is separated into a third flowthrough fraction and a third membrane fraction; and
wherein the cancer microparticles are enriched in at least one of the six membrane and flowthrough fractions.
